(19) United States
(12) Patent Application Publication
(10) Pub. No.: US 2017/0156708 A1
(43) Pub. Date: Jun. 8, 2017

(54) PHANTOM AND PACKAGED PHANTOM FOR EVALUATING PHOTOACOUSTIC MEASUREMENT APPARATUS

(71) Applicant: CANON KABUSHIKI KAISHA, Tokyo (JP)

(72) Inventors: Nobuhito Suehira, Tokyo (JP); Ryo Ogawa, Kawasaki-shi (JP); Takahiro Suita, Kawasaki-shi (JP); Hisafumi Ebisawa, Tokyo (JP)

(21) Appl. No.: 15/362,682

(22) Filed: Nov. 28, 2016

(30) Foreign Application Priority Data
Dec. 3, 2015 (JP) 2015-236744

(51) Int. Cl. A61B 8/00; G01N 29/30; G01N 21/17

(52) U.S. Cl. CPC A61B 8/587; G01N 21/17; G01N 29/30

(57) ABSTRACT

In the performance evaluation of a photoacoustic measurement apparatus using a phantom, an increase in the accuracy of the evaluation of the apparatus is improved. A phantom capable of being used to evaluate a photoacoustic measurement apparatus includes a recording unit having stored therein information regarding a temporal change in an optical characteristic or an acoustic characteristic of the phantom.

### BACKGROUND OF THE INVENTION

Field of the Invention

The present disclosure relates to a technique regarding a phantom and a packaged phantom for evaluating a photoacoustic measurement apparatus.

Description of the Related Art

In recent years, as one of optical imaging techniques, photoacoustic tomography (PAT) is discussed in Review of Scientific Instruments vol. 77 041101 2006. PAT is a technique for emitting pulse light to a specimen, such as living tissue, detecting photoacoustic waves generated by the specimen absorbing the energy of light propagated and diffused in the specimen, processing signals of the photoacoustic waves, and visualizing information related to optical characteristic values of the inside of the specimen.

Generally, to evaluate or calibrate the performance of an apparatus, a phantom simulating a specimen is used. In the phantom, a target of which the shape is known in advance is placed, and the performance of the apparatus is evaluated based on how the target is imaged. Such a phantom is discussed in the publication of Japanese Patent Application Laid-Open No. 2011-209691. In the publication of Japanese Patent Application Laid-Open No. 2011-209691, a phantom is made using a material having an optical characteristic and an acoustic characteristic close to those of human tissue. In a case where such a phantom is used to evaluate a photoacoustic tomography apparatus (a PAT apparatus) for measuring human tissue, it is possible to reduce the reflection of an acoustic wave at the interface between the apparatus and the phantom. Thus, erroneous evaluation can be reduced, which is preferable.

In the performance evaluation of a photoacoustic measurement apparatus using a phantom, however, further improvement is needed regarding an increase in the accuracy of the evaluation of the apparatus.

### SUMMARY OF THE INVENTION

According to an aspect of the present disclosure, a phantom capable of being used to evaluate a photoacoustic measurement apparatus includes a recording unit having stored therein information regarding a temporal change in an optical characteristic or an acoustic characteristic of the phantom.

According to another aspect of the present disclosure, a packaged phantom includes a phantom capable of being used to evaluate a photoacoustic measurement apparatus, and a recording unit having stored therein information regarding a temporal change in an optical characteristic or an acoustic characteristic of the phantom.

Further features will become apparent from the following description of exemplary embodiments with reference to the attached drawings.

### BRIEF DESCRIPTION OF THE DRAWINGS

FIG. 1 is a diagram of a first example illustrating a structure of a phantom.

FIGS. 2A, 2B, 2C, and 2D are diagrams illustrating data attached to the phantom.

FIG. 3 is a graph illustrating an example (an Arrhenius plot) where a temporal change is converted.

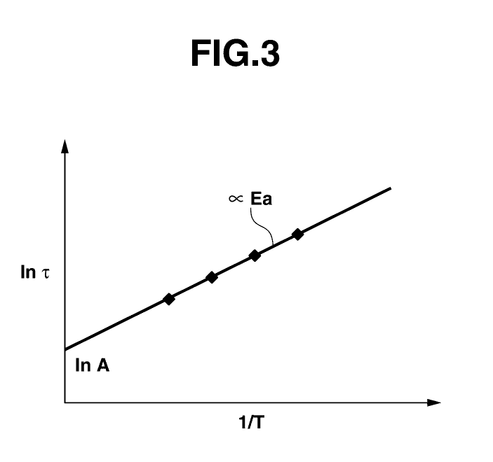

FIGS. 4A and 4B are diagrams illustrating an example of a photoacoustic measurement apparatus.

FIG. 5 is a diagram illustrating another example of a structure of a phantom.

FIG. 6 is a diagram illustrating an example of a structure of a phantom according to a second exemplary embodiment.

### DESCRIPTION OF THE EMBODIMENTS

Suitable exemplary embodiments of the present disclosure will be described below with reference to the drawings.

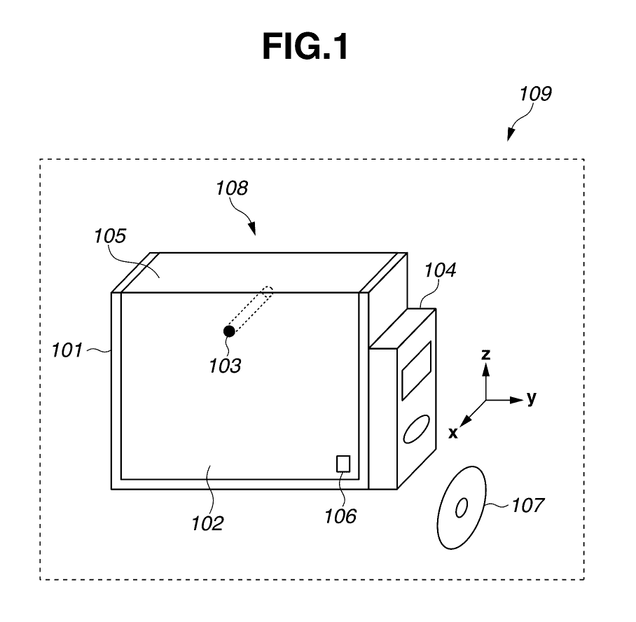

As illustrated in FIG. 1, as a phantom or a packaged phantom (to be described below) according to the present exemplary embodiment that is capable of being used for evaluation of a photoacoustic measurement apparatus, a phantom 108 or a packaged phantom 109 includes an integrated circuit (IC) tag 106 or a medium 107, such as a digital versatile disc (DVD) or a Universal Serial Bus (USB) medium, which is a recording unit. The recording unit has stored therein information regarding a temporal change in an optical characteristic or an acoustic characteristic of the phantom 108. Consequently, it is possible to evaluate the photoacoustic measurement apparatus with high accuracy. This is described below.

In a case where an apparatus is evaluated using a phantom, the performance of the apparatus is evaluated using a signal from a target 103 (described in detail below) of the phantom 108 and a measured image created based on the signal. However, in the characteristic of a phantom used for a photoacoustic measurement apparatus, a change (mainly deterioration) occurs due to hardening or alteration (discoloration) of the phantom by light emitted to the phantom, hydrolysis due to soaking the phantom in water, or the interaction of materials included in the phantom. Thus, to accurately evaluate the apparatus, it is necessary to take the deterioration of the phantom into account. Specifically, in a case the phantom which has been deteriorated is measured by the apparatus, the apparatus may be erroneously evaluated as requiring adjustment although the apparatus correctly measures information of the deteriorated phantom. That is, although the measurement result (the measured image) changes due to a change in the state of the phantom, the apparatus may be erroneously evaluated as the measurement performance of the apparatus has changed. Particularly, since an oxygen saturation measured by the photoacoustic measurement apparatus requires quantitative evaluation, if the apparatus is erroneously adjusted as described above, the quantitativity of the result of the subsequent specimen measurement is impaired. This leads to a major problem.

Thus, a recording unit having stored therein information regarding a temporal change in the optical characteristic or the acoustic characteristic of the phantom is provided in the main body of the phantom, or a package is formed of a set including the recording unit and the phantom, whereby it is possible to evaluate the photoacoustic measurement apparatus based on the information stored in the recording unit. As a result, it is possible to distinguish between a change in the measurement result according to a temporal change in the phantom and a change in the measurement result according to a change in the measurement performance of the photoacoustic measurement apparatus. This increases the accuracy of the performance evaluation of the photoacoustic measurement apparatus.

An example embodiment of the phantom or the packaged phantom is described below.

As illustrated in FIG. 1, the example phantom or the example packaged phantom includes a base material 102 and a target 103, which has an optical characteristic different from that of the base material 102. For example, in the case of a phantom simulating a breast, the base material 102 may simulate the optical characteristics and the acoustic characteristics of fat and a mammary gland, and the target 103 may simulate the optical characteristic and the acoustic characteristic of blood. To this end, for example, the base material 102 and the target 103 may be formed of a polyol and fillers capable of being dispersed in the polyol. Examples of the polyol include a polyether polyol, a polyester polyol, and a polycarbonate polyol. It is, however, more preferable to use a polyether polyol in view of a correlation regarding the sound propagation characteristic of human tissue. Normally, a polyol is in a liquid state. Thus, a curing agent is added to the polyol as needed, whereby the polyol resin cures into a solid. To approximate the sound propagation characteristics of the base material 102 and the target 103 to that of human tissue, an isocyanate compound may be used as the curing agent. Further, to approximate the light propagation characteristics of the base material 102 and the target 103 to that of human tissue, the equivalent scattering coefficients and the absorption coefficients of the base material 102 and the target 103 are adjusted by dispersing fillers. Examples of a filler having a light scattering property include an inorganic oxide, such as titanium oxide. Further, a pigment as a filler having a light absorption property may be used. Examples of the pigment include a black pigment, such as carbon black, a cyan pigment, such as copper phthalocyanine, a magenta pigment, such as a monoazo lake pigment or a monoazo pigment, and a yellow pigment, such as diarylide yellow.

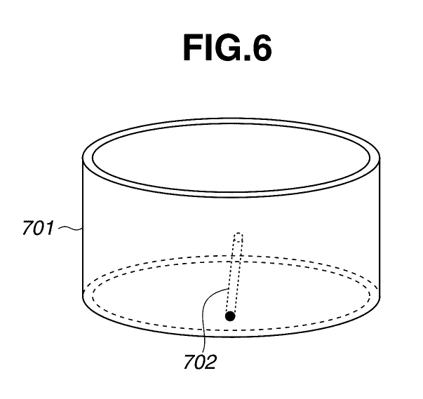

The phantom and the packaged phantom is not limited to the form in which the phantom and the packaged phantom include a base material and a target as described above. Alternatively, as will be described below with reference to FIG. 6, the phantom and the packaged phantom may include a target and a frame member in which the target is provided. The frame member may be formed of a transparent body, such as acrylic, so as not to absorb light and generate an unnecessary acoustic wave.

Examples of temporal deterioration information of the thus configured phantom include temporal deterioration information of an optical characteristic and temporal deterioration information of an acoustic characteristic, specifically, temporal deterioration information of the light attenuation characteristic of the base material 102 and temporal deterioration information of the light absorption characteristic of the target 103. These pieces of information have particularly great influence on a change in a measured image of the phantom and therefore are information particularly useful as temporal deterioration information. The acoustic characteristic is the speed of sound in the phantom or the sound pressure of an acoustic wave generated by the phantom.

As a recording unit for storing these pieces of information, an IC tag, a DVD, a USB medium, or the like can be used. The IC tag can be included in the main body of the phantom. The medium, such as the DVD and the USB medium, can be provided as an item included in a packaged formed of a set including the medium and the main body of the phantom.

Further, in an exemplary embodiment, an environment recording unit for recording information regarding the environment around the phantom may be further included. This is because the degree of progress of the deterioration of the phantom is greatly influenced by the environment state around the phantom. Thus, such information is recorded, whereby it is possible to understand the temporal change state of the phantom more accurately. Specifically, based on the information regarding the environment around the phantom recorded in the environment recording unit, the information regarding the temporal change stored in the recording unit is corrected, whereby it is possible to obtain more accurate temporal change information. For example, the recording unit for recording the temporal change in the phantom may have stored therein conversion information for correcting the information regarding the temporal change in the phantom based on the information regarding the environment around the phantom. Thus, it is possible to correct the temporal change information more easily.

In the phantom, as described above, deterioration, such as alteration, occurs due to the influence of heat or moisture. Thus, the information regarding the environment around the phantom may be temperature information and humidity information of the environment around the phantom.

Example embodiments will be described below.

An example phantom is described below with reference to the drawings.

### (Configuration of Phantom)

In a phantom 108 according to a first exemplary embodiment, a target 103, which has a columnar shape having a diameter of 1 mm, is placed in a base material 102 at a depth of 20 mm from a measurement surface 105. The measurement surface 105 is a surface with which a probe comes into contact. The other surfaces are covered with an outer frame 101 to maintain the shape of the phantom 108. Further, to the phantom 108, a recorder 104 is attached, which is an environment recording unit for monitoring a change in the environment around the phantom 108.

The target 103 is used to evaluate the initial sound pressure distribution of a photoacoustic apparatus. The target 103 has an absorption coefficient of 0.2 mm⁻¹, which is equivalent to blood. Meanwhile, the base material 102 of the phantom 108 has as optical constants an absorption coefficient of 0.005 mm⁻¹ and an equivalent scattering coefficient of 1 mm⁻¹, which are in the range of values of a living body. Further, in the base material 102 of the phantom 108, the speed of sound is 1450 m/s and an attenuation rate is 0.5 dB/cm MHz. The target 103 and the base material 102 are made of a polyol and fillers capable of being dispersed in the polyol. In the present exemplary embodiment, an isocyanate compound is added as a curing agent to a polyether polyol. As a filler having a light scattering property, titanium oxide is used. As a filler having a light absorption property, a carbon black pigment is used. Then, the compounding ratio between the fillers is adjusted to obtain the above characteristics.

The type of the target 103 is not limited to a target for evaluating initial sound pressure distribution. Alternatively, the target 103 may be intended to evaluate an oxygen saturation, evaluate a viewing angle, evaluate a resolution, or evaluate an optical resolution. In a case where an oxygen saturation is evaluated, a phantom including targets having oxygen saturations of 75% and 95% and each having a diameter of 1 mm can be used. These oxygen saturations can be obtained by preparing a pigment so that the ratio between the absorption coefficients of the targets is equivalent to the ratio between the oxygen saturations of the targets at wavelengths of 760 nm and 800 nm. To evaluate a viewing angle, a phantom including a ring-shaped target having an inner diameter of 10 mm and an outer diameter of 11 mm, for example, and having a light absorption coefficient can be used. Further, to evaluate a resolution, a nylon wire having a diameter of 0.1 mm and having an absorption coefficient may be used. To evaluate an optical resolution, two nylon wires having diameters of 0.3 and 0.5 mm may be placed parallel with each other with a space therebetween such that the space has a width equal to the thickness of each wire.

Further, the shape of the target 103 is not limited to a columnar shape as illustrated in FIG. 1. Alternatively, the target 103 may have a spherical shape or a ring shape. As a matter of course, a plurality of these targets can also be placed. A phantom, however, is often molded by placing a target in a mold and then pouring a base material into the mold. Thus, it is preferable to use a target having a linear shape, such as a column or a wire, because such a target can make it easier to manufacture the phantom.

### (Temporal Change Data)

If a phantom is appropriately manufactured, it is possible to produce phantoms having equivalent parameters with excellent reproducibility of design values (reproduce the same states (characteristics) in all the phantoms). In this case, data obtained by measuring one of the phantoms can be applied as it is as data of all the other phantoms. Thus, data of a temporal change to be attached to a phantom can be obtained in advance from an acceleration test for a phantom. As a method for the acceleration test, a phantom is placed in a thermostatic bath having constant temperature and humidity, and an image of the phantom is evaluated every 24 hours, for example. The thermostatic bath may be in an environment where, for example, the temperature is 40 to 70° C., and the humidity is 50% (an environment where deterioration is more likely to occur than in a normal use environment).

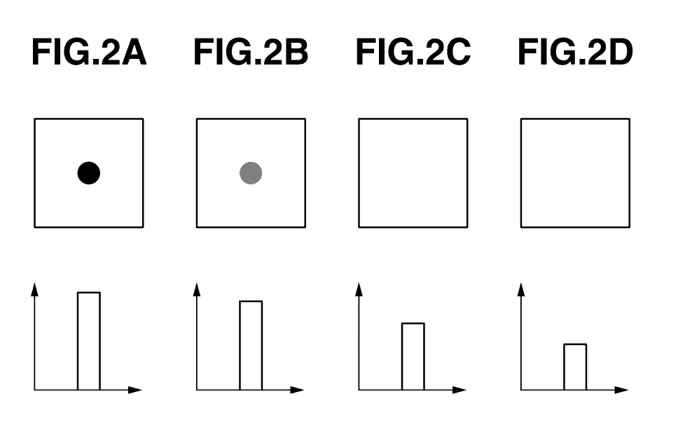

FIGS. 2A to 2D are diagrams each schematically illustrating a state of an image of the initial sound pressure distribution of a photoacoustic apparatus in a case where a phantom subjected to an acceleration test is measured by the photoacoustic apparatus. In each of FIGS. 2A to 2D, the upper part illustrates the contrast of the image of the initial sound pressure distribution, and the lower part illustrates a line profile of the sound pressure of the initial sound pressure distribution. FIGS. 2A to 2D represent the degree of the lapse of time (FIG. 2D represents the state where the most time elapses). As time elapses, the contrast changes (decreases). It is considered that this is because in a case where a target and a base material are formed of similar materials, the pigment of the target diffuses to the base material around the target with the lapse of time. Thus, it is considered that as a result, the contrast changes due to a temporal change in the optical characteristic of the phantom, specifically, a temporal change in the light absorption characteristic of the target. Further, in addition to this, a temporal change in the optical characteristic of the phantom, specifically, a temporal change in the light attenuation coefficient of the base material, occurs due to the discoloration of the entirety of the base material itself. Thus, it is considered that the contrast decreases also due to this temporal change. Such a change in the image is translated into an elapsed time in a reference environment, and the elapsed time is associated with data of the image and the numerical value of the sound pressure.

The reference environment has a temperature of 25° C. and a humidity of 40%, for example. The data can be converted into changes in shorter elapsed times by interpolating the data. As a method for the interpolation, the data may be represented by a curve, using linear interpolation or polynomial regression.

Further, the information (data) of the temporal change based on the above acceleration test to be attached to the phantom is recorded in a medium 107, such as a DVD or a USB medium. As a matter of course, the information (data) is recorded not only in this format. Alternatively, the data may be printed out. In the present application, a set including a phantom and a member in which information regarding the phantom is thus recorded (written) is referred to as a “packaged phantom” or a “phantom set.” The present invention is not limited to the form of a package (the form of a set). Alternatively, the data may be stored in an IC tag 106, which is provided in the phantom. That is, a unit for recording data may be provided in the main body of the phantom itself.

The data of the phantom may be data with respect to each material. In this case, each of the base material and the target is subjected to an acceleration test, and data of the acceleration test is attached to the phantom. In this case, the value of each member according to an elapsed time and apparatus parameters are further input, and simulation is performed, whereby it is possible to obtain an image in a case where the apparatus is evaluated based on the apparatus parameters at a certain time.

### (Apparatus Evaluation)

An example case where an apparatus is evaluated using an image of the initial sound pressure distribution as illustrated in FIGS. 2A to 2D, is described herein. In a case where an apparatus is evaluated using an image of the initial sound pressure distribution, the apparatus is evaluated based on the sound pressure and the resolution obtained from the gray scale of the image of the initial sound pressure distribution and a line profile. First, based on the state of use of the phantom (the time elapsed since the purchase of the phantom), an image corresponding to an elapsed time in the reference environment (e.g., an image of the phantom after a year of use) is selected as a reference image. Then, a measured image and a numerical value are compared with the selected reference image and a numerical value, respectively. As a result of the comparison, if there is a difference between these images, it is considered that a change may occur in the apparatus.

As a method for displaying a screen for evaluation, for example, a reference image and an actual measured image may be arranged and displayed next to each other. Alternatively, a cursor line may be provided on an acquired image according to an evaluation item, and the contrast of the acquired image and the contrast of a reference image on the line may be displayed as line profiles (the form in the lower parts of FIGS. 2A to 2D). Yet alternatively, the configuration may be such that the difference between a reference image and an actual measured image may be displayed. Further, only the numerical value of the percentage of deterioration as compared with the time of shipment of the phantom may be displayed.

### (Conversion of Elapsed Time)

The deterioration of the phantom depends not only on the elapsed time but also largely on the use environment. Thus, the elapsed time may be calculated by, according to the state of use of the phantom, converting an actual elapsed time into an elapsed time in a case where the phantom is used in the reference environment. In this case, the recorder 104, which is the environment recording unit, measures the temperature of the use environment (the environment around the phantom). The temperature is recorded every 30 minutes, for example. As a matter of course, not only the temperature but also the humidity may be measured. Further, the recorded data may be able to be transferred wirelessly to the apparatus. Based on the information of the environment around the phantom, the temporal change information is corrected using, for example, the conversion information stored in the IC tag 106, which is the recording unit for recording the temporal change information of the phantom. An example of the correction of the temporal change information using the conversion information is described below.

In a case where the elapsed time of the phantom in the actual use environment is converted into an elapsed time in a case where the phantom is used in the reference environment, for example, the Arrhenius model can be used. The Arrhenius model is represented by the following formula 1 using a natural logarithm In:

$$ln τ = ln A + \frac{{E}_{a}}{RT} , \qquad ( 1 )$$

where τ is a life, A is a coefficient, E_{d} is the activation energy of a target a, R is a gas constant, and T is an absolute temperature.

The life is measured by changing the temperature, whereby it is possible to determine A and Eₐ. Here, T may not be the life, and may be the time in which a certain change occurs. That is, τ may be the time in which an absorption coefficient becomes smaller than a desired value. Thus, it is possible to obtain a coefficient in this case. As a result, if a time τ₂ elapses at a temperature T₂, then in the case of a temperature T₁ (e.g., 25° C.), the elapsed time of the phantom can be converted into an elapsed time as the following formula 2:

$${τ}_{1} = {τ}_{2} exp ( \frac{{E}_{a}}{{RT}_{1}} - \frac{{E}_{a}}{{RT}_{2}} ) . \qquad ( 2 )$$

Consequently, based on the environment such as the temperature measured by the recorder 104, it is possible to convert the elapsed time of the phantom into an elapsed time in the reference environment.

### (Phantom Management)

Generally, in a case where an apparatus is used in a hospital, the apparatus is often managed for 24 hours in a constant temperature and humidity environment. Even in such a case, the phantom may be stored in a case designed for the phantom after using the phantom. The case designed for the phantom is covered with a material, such as duralumin, whereby it is possible to shield the phantom from light. The case for storing the phantom includes a mechanism for fixing the phantom. A buffer material is further placed in the case, whereby it is possible to protect the phantom from the impact of a fall. Further, a desiccant is placed in the case, whereby it is possible to maintain a constant humidity inside the case and obtain a storage state in the reference environment. Thus, the accuracy of the estimation of temporal deterioration is increased. As a matter of course, in a case where the use environment or the storage environment is different from the reference environment, the elapsed time may be converted into an elapsed time in the reference environment as described above.

In the above manner, even in a case where the state of a phantom changes, it is possible to accurately evaluate an apparatus.

Next, a description is given of a series of operations for calibrating a photoacoustic measurement apparatus for a breast using the phantom, together with the configuration of the photoacoustic measurement apparatus for a breast. A description is given below of, as the measurement of a breast by the photoacoustic measurement apparatus, the operation of calibrating the apparatus based on the measurement of an oxygen saturation using the phantom for the purpose of increasing the accuracy of the measurement of an oxygen saturation.

### (Photoacoustic Apparatus)

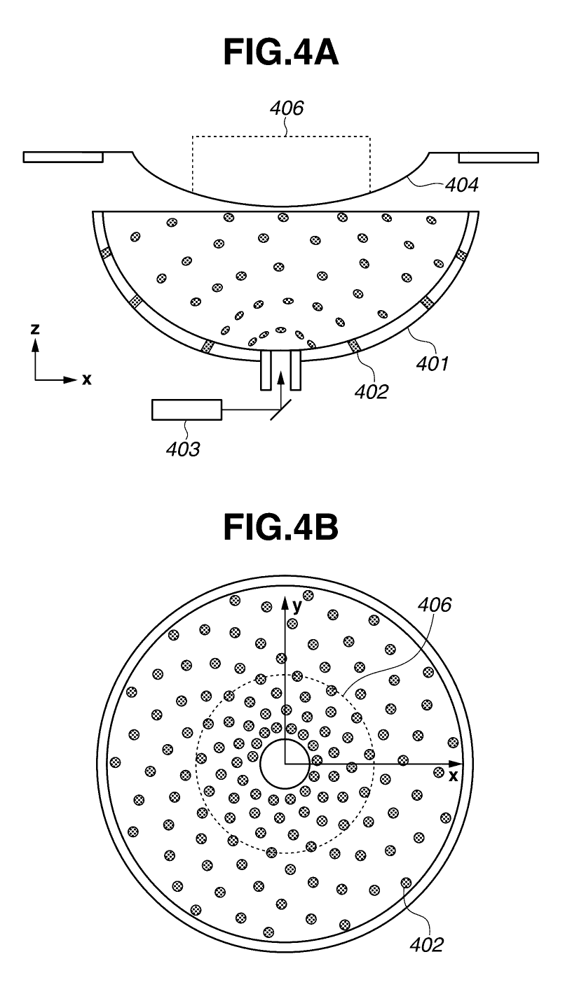

FIGS. 4A and 4B are diagrams illustrating a photoacoustic apparatus for measuring a breast. FIG. 4A is a cross-sectional view illustrating the configuration of a portion, in the photoacoustic measurement apparatus, for holding a specimen. Further, FIG. 4B is a top view of the portion for holding a specimen. Reception elements 402 receives an acoustic wave, and 512 of the reception elements 402 are placed spirally along a hemispherical surface on a hemispherical container (a unit for supporting the reception elements) 401. A specimen is placed on a holding member 404. In FIGS. 4A and 4B, the position of a phantom 406 which is used in this measurement is indicated by a dotted line.

Between the hemispherical container 401 and the holding member 404, an acoustic matching layer, such as water, is interposed. Further, although the phantom 406 is placed in the holding member 404, a matching layer, such as water, is also placed as needed in the holding member 404 to prevent air from entering the path of an acoustic wave. The holding member 404 is formed of polyethylene terephthalate. Further, in the hemispherical container 401, a space is provided through which measurement light from a light emission unit 403 passes. Then, it is possible to emit measurement light to the specimen or the phantom 406 from a negative z-axis direction. Further, it is possible to change the position of the hemispherical container 401 using an XY stage (not illustrated). The light emission unit 403 emits pulse light to the specimen or the phantom 406 while scanning the XY stage, and the reception elements 402 detects a generated acoustic wave. Data of the detection result is reconfigured, whereby it is possible to obtain a three-dimensional photoacoustic image.

### (Light Emission Unit)

The light emission unit 403 generates pulse light to be emitted to the specimen or the phantom 406 and emits the generated pulse light. A light source for generating pulse light may be a laser light source to obtain large output. The present invention, however, is not limited to this. Alternatively, a light-emitting diode or a flash lamp may be used instead of the laser. In a case where the laser is used, various lasers, such as a solid-state laser, a gas laser, a pigment laser, and a semiconductor laser can be used.

To effectively generate a photoacoustic wave, it is necessary to emit light in a sufficiently short time according to the heat characteristic of the specimen or the phantom 406. In a case where the specimen is a living body, a suitable pulse width of pulse light generated by the light source is about 10 to 50 nanoseconds. Further, the wavelength of the pulse light may be a wavelength at which light is propagated into the specimen. Specifically, in the case of a living body, the wavelength of the pulse light is between 700 nm or more and 1100 nm or less. In the present exemplary embodiment, a titanium-sapphire laser, which is a solid-state laser, is used, and wavelengths of 760 and 800 nm are used to measure oxygen saturations.

### (Reception Elements for Photoacoustic Measurement)

The reception elements 402 receive a photoacoustic wave. In the present exemplary embodiment, capacitive micromachined ultrasonic transducers (CMUTs) are used. Each of the reception elements 402 has an opening having a diameter of 3 mm and has a band of 0.5 to 5 MHz as a reception characteristic. Sampling is performed 2048 times at a sampling frequency of 50 MHz, and each sample is obtained in 12-bits.

### (Phantom)

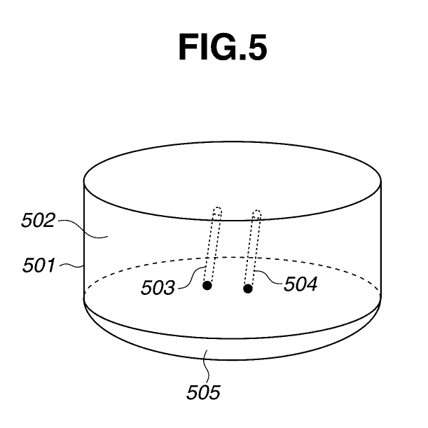

FIG. 5 is a diagram illustrating the structure of a phantom 501, which is used to measure oxygen saturations. A measurement surface 505 of the phantom 501 is designed to fit a curved surface of the holding member 404. Targets 503 and 504, which have oxygen saturations of 75% and 95%, respectively, are placed away from each other at a depth of 20 mm from the measurement surface 505 such that the space between the targets 503 and 504 has a width of 10 mm. Each target has a diameter of 1 mm as its thickness.

In the targets 503 and 504, a pigment is adjusted so that the ratio between the absorption coefficients at two wavelengths of 760 nm and 800 nm is the same as the ratio between the oxygen saturations of 75% and 95%.

Further, in the phantom 501, an image of initial sound pressure distribution and images of oxygen saturations that change with the lapse of time are prepared in advance. These images are obtained by performing an acceleration test on a phantom having the same configuration as that of the phantom 501. As described above, the images may be recorded in advance in a medium provided together with the phantom 501, or this information may be stored in an IC tag provided in the phantom 501.

### (Evaluation of Photoacoustic Measurement Apparatus)

A photoacoustic measurement apparatus is evaluated by comparing an actual measured image of a phantom acquired by the photoacoustic measurement apparatus, with an image (e.g. obtained through simulation) of the phantom according to the elapsed time prepared in advance. First, based on the state of use of the phantom (the time elapsed since the purchase of the phantom), images corresponding to an elapsed time in the reference environment (e.g., images corresponding to the phantom after a year of use) are selected as reference images. In this case, an image of an oxygen saturation and an image of initial sound pressure distribution at each wavelength are selected together. Then, an actual measured image of the initial sound pressure distribution at each wavelength and an actual measured image of the oxygen saturation are compared with the respective reference images selected first. If the proportion of the difference between the contrasts or the luminance values of compared images is within ±1%, it is possible to determine that there is no abnormality in the photoacoustic measurement apparatus. If, on the other hand, for example, the initial sound pressure distribution at each wavelength changes in the same proportion, it is determined that there may be abnormality in the transmittance of the optical system.

In a case where selected reference images (images obtained by simulation) are compared with measured images, the comparison can be made for the ratio between the absorption coefficients and the oxygen saturations of a plurality of targets (the ratio between the absorption coefficients and the oxygen saturations of the targets 503 and 504). The use of the ratio between a plurality of targets as described above has the advantage that it is not necessary to measure all the parameters of the targets. That is, if, as in the targets 503 and 504 provided in a phantom for calibrating a photoacoustic measurement apparatus using the measurement results of oxygen saturations, only the absorption coefficients at respective wavelengths are different from each other, and other members are similar in the phantom, the difference in influence of deterioration between other elements is common and therefore can be ignored. Thus, it is not necessary to obtain (compare) the absolute values of elements with high accuracy. Thus, it is possible to evaluate the apparatus by simple comparison.

As described above, even in a case where the state of a phantom changes, it is possible to accurately evaluate an apparatus.

Similarly to the first exemplary embodiment, a phantom according to a second exemplary embodiment is used to evaluate a photoacoustic measurement apparatus for a breast. The second exemplary embodiment, however, is different from the first exemplary embodiment in that the phantom includes an outer frame and a target (does not include a base material). In the present exemplary embodiment, since a base material is not included, it is possible to estimate the influence of a temporal change more easily and more accurately.

FIG. 6 is a diagram illustrating the phantom according to the present exemplary embodiment. This phantom includes an outer frame 701 and a photoacoustic measurement target 702, which is installed in the outer frame 701. The outer frame 701 is formed of a transparent body, such as acrylic, and does not absorb light. Thus, the outer frame 701 hardly produces a sound. The photoacoustic measurement target 702 is fixed through a hole in the outer frame 701. The photoacoustic measurement target 702 has a diameter of 1 mm.

In measurement, a liquid is put into the holding member 404, and the photoacoustic measurement target 702 is located in the liquid, whereby the target 702 is measured in the state where a gap (an acoustic impedance mismatch surface) is not formed on the propagation path of a photoacoustic wave from the photoacoustic measurement target 702 to the reception elements 402. As the liquid, water or a dilution of a fat emulsion (a liquid containing soybean oil) for use in the nutritional management of veins can be used.

In this case, only the photoacoustic measurement target 702 needs to be subjected to an acceleration test to obtain a desired image (temporal change information). Thus, it is possible to estimate a temporal change in the phantom more accurately. As a result, it is possible to evaluate a photoacoustic measurement apparatus more easily and more accurately.

In the above manner, even in a case where the state of a phantom changes, it is possible to accurately evaluate an apparatus.

In the above exemplary embodiment, a description has been given of a technique for calibrating an apparatus using temporal change information attached to a phantom. Alternatively, the apparatus may be calibrated using not only the information attached to the phantom, but also temporal change information obtained (acquired) via a network. That is, it is also possible to accurately calibrate the apparatus by the following method. Temporal change information is acquired, regardless of the method for obtaining the temporal change information, and a reference image calculated based on the acquired temporal change information is compared with an image obtained by actually measuring the phantom, thereby determining a change in the apparatus and calibrating the apparatus.

According to the present disclosure, it is possible to evaluate a photoacoustic measurement apparatus with high accuracy.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-236744, filed Dec. 3, 2015, which is hereby incorporated by reference herein in its entirety.

What is claimed is:

1. A phantom capable of being used to evaluate a photoacoustic measurement apparatus, the phantom comprising:
a recording unit having stored therein information regarding a temporal change in an optical characteristic or an acoustic characteristic of the phantom.

2. The phantom according to claim 1, further comprising:
a base material and a target having an optical characteristic different from an optical characteristic of the base material.

3. The phantom according to claim 1, further comprising:
a target and a frame member in which the target is installed.

4. The phantom according to claim 1, wherein the information regarding the temporal change is temporal change information of the optical characteristic of the phantom.

5. The phantom according to claim 2, wherein the information regarding the temporal change is temporal change information of a light absorption characteristic of the target.

6. The phantom according to claim 2, wherein the information regarding the temporal change is temporal change information of a light attenuation characteristic of the base material.

7. The phantom according to claim 1, further comprising:
an environment recording unit configured to record information regarding an environment around the phantom.

8. The phantom according to claim 7, wherein the recording unit further has stored therein conversion information for, based on the information regarding the environment around the phantom recorded in the environment recording unit, correcting the information regarding the temporal change stored in the recording unit.

9. The phantom according to claim 8, wherein the information regarding the environment around the phantom is temperature information of the environment around the phantom.

10. The phantom according to claim 8, wherein the information regarding the environment around the phantom is humidity information of the environment around the phantom.

11. The phantom according to claim 8, wherein the information regarding the environment around the phantom is temperature information and humidity information of the environment around the phantom.

12. A packaged phantom comprising:
a phantom capable of being used to evaluate a photoacoustic measurement apparatus; and
a recording unit having stored therein information regarding a temporal change in an optical characteristic or an acoustic characteristic of the phantom.

13. The packaged phantom according to claim 12, further comprising:
a base material and a target having an optical characteristic different from an optical characteristic of the base material.

14. The packaged phantom according to claim 12, further comprising:
a target and a frame member in which the target is installed.

15. The packaged phantom according to claim 12, wherein the information regarding the temporal change is temporal change information of the optical characteristic of the phantom.

16. The packaged phantom according to claim 13, wherein the information regarding the temporal change is temporal change information of a light absorption characteristic of the target.

17. The packaged phantom according to claim 13, wherein the information regarding the temporal change is temporal change information of a light attenuation characteristic of the base material.

18. The packaged phantom according to claim 12, further comprising:
an environment recording unit configured to record information regarding an environment around the phantom.

19. The packaged phantom according to claim 18, wherein the recording unit further has stored therein conversion information for, based on the information regarding the environment around the phantom recorded in the environment recording unit, correcting the information regarding the temporal change stored in the recording unit.

20. The packaged phantom according to claim 19, wherein the information regarding the environment around the phantom is temperature information of the environment around the phantom.

21. The packaged phantom according to claim 19, wherein the information regarding the environment around the phantom is humidity information of the environment around the phantom.

22. The packaged phantom according to claim 19, wherein the information regarding the environment around the phantom is temperature information and humidity information of the environment around the phantom.
